(19) United States
(12) Patent Application Publication
(10) Pub. No.: US 2004/0002155 A1
(43) Pub. Date: Jan. 1, 2004

(54) Endonuclease VII cloning method

(75) Inventors: Alan L. Greener, San Diego, CA (US); Lisa Joy Hexdall, San Diego, CA (US); Carsten-Peter Carstens, San Diego, CA (US); Joseph A. Sorge, Wilson, WY (US)

Correspondence Address: PALMER & DODGE, LLP, KATHLEEN M. WILLIAMS / STR, 111 HUNTINGTON AVENUE, BOSTON, MA, 02199, US

(73) Assignee: Stratagene

(21) Appl. No.: 10/180,174

(22) Filed: Jun. 26, 2002

(51) Int. Cl. C12N 15/85; C12N 15/74; C12Q 1/68; C12N 1/21; C12N 5/06

(52) U.S. Cl. 435/455; 435/472; 435/325; 435/252.3

(57) ABSTRACT

The subject invention provides for a strain of host cells that contains a temperature sensitive variant of the gene encoding the endonuclease VII from phage T4. Using this host strain, the invention features a novel cloning method that selects for PCR products that are devoid of PCR-generated mutations.

### BACKGROUND

PCR amplification is known to create mutations at a much higher rate than in vivo propagation (replication) of DNA. The mutation rate will vary with PCR conditions, choice of enzyme, number of replication cycles, etc. After 30 rounds of PCR amplification, it is estimated that between 2% and 10% of the molecules contain at least one mutation. Thus, when PCR amplified DNA is cloned into a plasmid vector, 2-10% of the cloned inserts will contain mutations. Depending on the downstream application, it is often necessary to sequence the amplified DNA prior to further experimentation.

There is therefore a need in the art for methods that select for PCR amplification products that do not have PCR induced mutations.

### SUMMARY OF THE INVENTION

The subject invention provides for a host strain that contains a temperature sensitive variant of the gene encoding the endonuclease VII from phage T4, e.g., the nucleic acid sequence of SEQ ID NO: 1, encoding the amino acid sequence of SEQ ID NO: 2. The present invention also provides a method for the selective cloning of amplified DNAs with a reduced amount of PCR induced mutations.

In one embodiment, the invention provides for a host cell for selectively cloning homoduplex DNA molecules. The host cell is competent, and contains a heterologous gene encoding a conditionally expressed and/or conditionally active resolvase, and the host cell can be maintained under conditions where the resolvase is not expressed and/or is repressed and propagated in inactive form. The host cell can be eukaryotic or prokaryotic. The resolvase can be a eukaryotic resolvase, or a bacteriophage resolvase, e.g., bacteriophage T4 endonuclease VII, e.g. a temperature sensitive mutant of bacteriophage T4 endonuclease VII, e.g., the temperature sensitive mutant of bacteriophage T4 endonuclease VII, e.g., SEQ ID NO: 2. The resolvase can be an enzyme the expression of which is completely repressed under certain conditions.

The invention also features a method of selectively cloning homoduplex nucleic acid molecules, where the method includes providing one or more host cells containing a heterologous gene encoding a conditionally expressed and/or conditionally active resolvase, transforming the host cells with cloned nucleic acid molecules, and then maintaining the transformed host cells so that the resolvase is expressed and active, and destroys the cloned heteroduplex molecules, leaving the homoduplex molecules.

The method can further include denaturing and renaturing the nucleic acid molecules before ligation. The method can also include, after action of the resolvase, infecting the cells with helper phage to rescue the homoduplex cloned nucleic acid molecules. The method can also include maintaining the host cells under conditions that prohibit the expression of the resolvase.

In another aspect, the invention features a method of selectively cloning amplified nucleic acid molecules possessing a reduced number of mutations, where the method includes providing one or more host cells containing a heterologous gene encoding a conditionally expressed and/or conditionally active resolvase, transforming the host cells with cloned amplified nucleic acid molecules, and then maintaining the transformed host cells under conditions suitable for expression of resolvase, so that the cloned heteroduplex molecules are destroyed by the resolvase, leaving the homoduplex cloned molecules.

The method can include the further step of denaturing and renaturing the nucleic acid molecules before ligation. The method can include the further step of, after action of the resolvase, infecting the cells with helper phage to rescue the homoduplex ligated nucleic acid molecules. The method can also include the further step of maintaining the host cells under conditions that prohibit the expression of the resolvase. The host cell can be eukaryotic or prokaryotic. The cloning vector can be a phagemid cloning vector. The conditionally expressed resolvase can be a eukaryotic resolvase or a bacteriophage resolvase, e.g., bacteriophage T4 Endonuclease VII, e.g., a temperature sensitive mutant of bacteriophage T4 Endonuclease VII, e.g., the temperature sensitive mutant of bacteriophage Endonuclease VII, e.g., the protein of SEQ ID NO: 2. The resolvase can be an enzyme the expression of which is completely repressed under certain conditions.

The pool of cloned nucleic acid molecules can contain both hetero- and homoduplex nucleic acids, and the heteroduplex molecules are destroyed by the resolvase in the methods.

In an additional aspect, the invention features a kit comprising a competent host cell containing a heterologous gene encoding a conditionally expressed resolvase, and packaging materials for same. The host cell can be maintained under conditions under which the resolvase is expressed. The kit can also contain a host cell containing an expression vector encoding such a resolvase, for example the temperature sensitive mutant of bacteriophage T4 Endonuclease VII of SEQ ID NO: 2, or the host cell can contain an isolated DNA (e.g., SEQ ID NO: 1) encoding the temperature sensitive mutant of bacteriophage T4 Endonuclease VII of SEQ ID NO: 2.

In any of the above kits, the resolvase can be an enzyme the expression of which is completely repressed under certain conditions. The kits can also contain other reagents, such as PCR reagents, and packaging materials.

The invention also features an isolated protein of a temperature sensitive mutant of bacteriophage T4 Endonuclease VII, where the isolated protein has the amino acid sequence of SEQ ID NO: 2, and an isolated nucleic acid encoding such a mutant, where the isolated nucleic acid has the sequence of SEQ ID NO: 1.

By the term “heteroduplex” is meant a structure formed between two annealed, complementary nucleic acid strands (e.g., the annealed strands of test and reference nucleic acids) in which one or more nucleotides in the first strand are unable to appropriately base pair with those in the second opposing, complementary strand because of one or more mismatches. Examples of different types of heteroduplexes include those that exhibit an exchange of one or several nucleotides, and insertion or deletion mutations, each of which is disclosed in Bhattacharyya and Lilley, Nucl. Acids. Res. 17: 6821 (1989). The term “complementary,” as used herein, means that two nucleic acids, e.g., DNA or RNA, contain a series of consecutive nucleotides which are capable of forming matched Watson-Crick base pairs to produce a region of double-strandedness. Thus, adenine in one strand of DNA or RNA pairs with thymine in an opposing complementary DNA strand or with uracil in an opposing complementary RNA strand. The region of pairing is referred to as a “duplex.” A duplex may be either a homoduplex or a heteroduplex. In a preferred embodiment, DNA is subjected to PCR, heat denatured and reannealed to generate homoduplexes and heteroduplexes.

Mismatch, as used herein, refers to a duplex in which one or more of the following is present: (1) DNA nucleotide pairing other than adenosine-thymine or guanine-cytosine occurs, e.g., nucleotide paring such as adenosine-cytosine, adenosine-guanine, adenosine-adenosine, thymine-cytosine, thymine-guanine, thymine-thymine, guanine-guanine, or cytosine-cytosine occurs; (2) a deletion or insertion of one or more DNA nucleotides on one strand as compared to the other complementary strand occurs, e.g., a deletion of 1, 2, 5, 10, 15, or more nucleotides or an insertion of 1, 2, 5, 10, 15, or more nucleotides occurs. DNA mismatches may arise from DNA replication errors, mutagenesis, deamination of 5-methylcytosine, and DNA recombination.

A “mutation”, as used herein, refers to a nucleotide sequence change (i.e., a nucleotide substitution, deletion, or insertion) in an isolated nucleic acid relative to a reference nucleic acid. In one embodiment, the reference nucleic acid is a template-specific nucleic acid used in an amplification reaction.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome-binding site, often along with other sequences. Eukaryotic cells are known to utilize promoters, enhancers, and termination and polyadenylation signals.

The terms “transform” and “transfect” as used herein refer to the introduction of foreign DNA into prokaryotic or eukaryotic cells. Transformation of prokaryotic cells may be accomplished by a variety of means known to the art including the treatment of host cells with various salt solutions or nonionic compounds (e.g., CaCl₂) to render the cells competent, electroporation treatment, etc. Transfection of eukaryotic cells may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

Resolvases are enzymes that process recombinational intermediates. They have the secondary effect of acting on mismatched DNA, which in some respects resembles a recombinational intermediate. “Resolvase”, as used herein, refers to an enzyme that cleaves a nucleic acid as the result of the presence of a distortion in a duplex, e.g., a bend, kink or other DNA deviation, e.g., a DNA mismatch, e.g., a single base pair substitution, insertion or deletion, in many different organisms, including bacteria, phage, yeast, and mammals, e.g., humans. The enzyme exerts its effect by mismatch-dependent cleavage i.e. cleavage of at least one DNA strand, close to the site of DNA distortion, e.g., a DNA mismatch.

Examples of resolvases include, without limitation, T4 endonuclease VII, Saccharomyces cerevisiae Endo X1, Endo X2, or Endo X3 (Jensch et al., EMBO J. 8:4325, 1989), T7 endonuclease I, E. coli MutY (Wu et al., Proc. Natl. Acad. Sci. USA 89:8779-8783, 1992), mammalian thymine glycosylase (Wiebauer et al., Proc. Natl. Acad. Sci. USA 87:5842-5845, 1990), topoisomerase I from human thymus (Yeh et al., J. Biol. Chem. 266:6480-6484, 1991; Yeh et al., J. Biol. Chem. 269:15498-15504, 1994), deoxyinosine 3′ endonuclease (Yao and Kow, J. Biol. Chem. 269:31390-31396, 1994) and Mus81 (Boddy et al., 2001, Cell 107:537-48; Chen et al., Mol. Cell. 8:1117-27, 2001). In preferred embodiments, the resolvase is isolated from a bacteriophage, e.g., bacteriophage T3, T4 or T7. In another preferred embodiment, the resolvase according to the invention is a mutated form (nucleic acid sequence, SEQ ID NO: 1; amino acid sequence SEQ ID NO: 2; FIG. 1) of the wild type endonuclease VII of phage T4 (GenBank Accession No.: X12629; nucleic acid sequence, SEQ ID NO: 3; amino acid sequence SEQ ID NO: 4; FIG. 2).

“Mismatch-dependent cleavage”, as used herein, refers to a characteristic of an enzyme, e.g., a resolvase. An enzyme has a mismatch-dependent cleavage activity if it cleaves at a mismatch, at a significantly higher rate, than it would cleave a corresponding perfectly matched sequence. In preferred embodiments, an enzyme with a mismatch-dependent cleavage is at least about 5%, 15%, 25%, 50%, 75% or 100% more efficient at cleaving at a mismatch than at a corresponding perfectly matched sequence.

As used herein, “conditionally expressed” and “conditionally active” refers to the expression of a mutant protein, which is only present and functional under permissive conditions.

In one embodiment, the mutant protein is “conditionally expressed”, that is, the protein is only produced under permissive conditions. Under non-permissive conditions, the protein is not produced, and is not present.

In another embodiment, the mutant protein is “conditionally active”, that is, the gene encoding the protein has a temperature sensitive mutation that renders the protein non-functional (i.e., inactive) at under permissive conditions, i.e., a permissive temperature. In one such embodiment, the gene encoding the mutant protein has a nonsense mutation that results in the translation of a truncated protein. In a preferred embodiment, “conditionally-expressed” or “conditionally active” refer to the expression of a temperature sensitive mutant T4 endonuclease VII protein that is active and functional but also lethal to the host cells at 25° C., for example, the temperature sensitive mutant T4 endonuclease VII protein, SEQ ID NO: 2, encoded by SEQ ID NO: 1.

A “host cell” is a cell which has been transformed or transfected, or is capable of transformation or transfection by an exogenous polynucleotide sequence. Host cells can be prokaryotic or eukaryotic, mammalian, plant, or insect, and can exist as single cells, or as a collection, e.g., as a culture, or in a tissue culture, or in a tissue or an organism. In a preferred embodiment, prokaryotic host cells are bacteria that harbor the F′ episome that enables f1 phage rescue as described herein. Host cells can also be derived from normal or diseased tissue from a multicellular organism, e.g., a mammal. Host cell, as used herein, is intended to include not only the original cell which was transformed with a nucleic acid, but also descendants of such a cell, which still contain the nucleic acid.

As used herein, “a mixture of DNA molecules” refers to DNA molecules which, when aligned, may vary in sequence at one or more positions or at no positions. In a preferred embodiment, the “mixture of DNA molecules” refers to DNA that was amplified by the polymerase chain reaction.

As used herein, the term “amplified”, when applied to a nucleic acid sequence, refers to a process whereby one or more copies of a particular nucleic acid sequence is generated from a template nucleic acid, preferably by the method of polymerase chain reaction (Mullis and Faloona, 1987, Methods Enzymol. 155:335). “Polymerase chain reaction” or “PCR” refers to an in vitro method for amplifying a specific nucleic acid template sequence. The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, thermostable DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers” or “a set of PCR primers” can comprise two, three, four or more primers. Other methods of amplification include, but are not limited to, ligase chain reaction (LCR), polynucleotide-specific based amplification (NSBA), or any other method known in the art.

As used herein, “nucleic acid polymerase” refers to an enzyme that catalyzes the polymerization of nucleotides. Generally, the enzyme will initiate synthesis at the 3′-end of the primer annealed to a nucleic acid template sequence, and will proceed in the 5′-direction along the template. “DNA polymerase” catalyzes the polymerization of deoxynucleotides. Known DNA polymerases include, for example, Pyrococcus furiosus (Pfu) DNA polymerase, E. coli DNA polymerase I, T7 DNA polymerase, Thermus thermophilus (Tth) DNA polymerase, Bacillus stearothermophilus DNA polymerase, Thermococcus litoralis (Tli) DNA polymerase (also referred to as Vent DNA polymerase), Thermotoga maritima (UlTma) DNA polymerase, Thermus aquaticus (Taq) DNA polymerase, and Pyrococcus GB-D (PGB-D) DNA polymerase. The polymerase activity of any of the above enzyme can be defined by means well known in the art. One unit of DNA polymerase activity, according to the subject invention, is defined as the amount of enzyme which catalyzes the incorporation of 10 nmoles of total dNTPs into polymeric form in 30 minutes at 72° C.

As used herein, “thermostable” refers to an enzyme which is stable and active at temperatures as great as preferably between about 90-100° C. and more preferably between about 70-98° C. as compared to a non-thermostable form of an enzyme with a similar activity that are typically denatured at such elevated temperatures. For example, a representative thermostable nucleic acid polymerase isolated from Thermus aquaticus (Taq) is described in U.S. Pat. No. 4,889,818 and a method for using it in conventional PCR is described in Saiki et al. (1988, Science 239:487). Another representative thermostable nucleic acid polymerase isolated from P. furiosus (Pfu) is described in Lundberg et al. (1991, Gene 108:1-6). Additional representative temperature stable polymerases include, e.g., polymerases extracted from the thermophilic bacteria Thermusflavus, Thermus ruber, Thermus thermophilus, Bacillus stearothermophilus (which has a somewhat lower temperature optimum than the others listed), Thermus lacteus, Thermus rubens, Thermotoga maritima, or from thermophilic archaea Thermococcus litoralis, and Methanothermus fervidus.

As used herein, a “PCR ligation mix” refers to the reaction mix after ligation has occurred in which an amplified DNA fragment is ligated to a recombinant DNA vector that is capable of autonomous replication within a host cell. In a preferred embodiment, the recombinant DNA vector is a phagemid.

As used herein, “competent” cells refers to host cells that are primed for the uptake of nucleic acids. Competent cells are treated to make their cell membranes more permeable in order to facilitate the entry of exogenous nucleic acids.

The term “recombinant DNA vector” as used herein refers to DNA sequences containing a desired coding sequence and appropriate DNA sequences necessary for the expression of the operably linked coding sequence in a particular host organism. DNA sequences necessary for expression in prokaryotes include a promoter, optionally an operator sequence, a ribosome-binding site and possibly other sequences. Eukaryotic cells are known to utilize promoters, polyadenylation signals and enhancers.

As used herein, “helper phage” refers to a normal wild-type version of the phage, which typically grows along with a specialized phage such as a phagemid (Bluescript, pUC and the like) and supplies whatever functions are necessary for generating phage particles. In one embodiment, the “helper phage” is M13KO7, a M13 phage that is able to replicate in E. coli in the absence of phagemid DNA. In the presence of a phagemid bearing a wild-type M13 or f1 origin, single-stranded phagemid is packaged preferentially and secreted into the culture medium.

As used herein, “rescue” refers to the recovery of homoduplex DNA that was not degraded according to the invention, by one or more resolvases within a host cell.

As used herein, “reducing the amount of DNA heteroduplexes” refers to a decrease in the number DNA heteroduplexes in a mixture of DNA molecules as a result of enzymatic cleavage by T4 endonuclease VII in a host cell. In a preferred embodiment, the decrease in the amount of DNA heteroduplexes is at least 75%, preferably 90%, more preferably 99% and most preferably 100% as compared to the amount of DNA heteroduplexes in a mixture of DNA molecules in the absence of enzymatic cleavage by T4 endonuclease VII in a host cell.

As used herein, “maintaining” the host cells refers to those experimental conditions of IPTG induction, competence to DNA transformation and non-permissive temperature i.e. high temperature that allows cell viability, expression of non-functional T4 endonuclease VII, digestion of heteroduplex ligated DNA and rescue of homoduplex DNA with f1 helper phage before the onset of host cell death.

### BRIEF DESCRIPTION OF THE DRAWINGS

FIG. 1 is a diagram showing the nucleic acid (SEQ ID NO: 1) and amino acid (SEQ ID NO: 2) sequences of the temperature-sensitive mutant T4 endonuclease VII.

FIG. 2 is a diagram showing the nucleic acid (SEQ ID NO: 3) and amino acid (SEQ ID NO: 4) sequences of the wild-type T4 endonuclease VII.

FIG. 3 is a diagram showing the alignment of the nucleic acid sequences of the temperature-sensitive mutant T4 endonuclease VII nucleic acid sequence (SEQ ID NO: 1) and the wild type T4 endonuclease VII nucleic acid sequence (SEQ ID NO: 3), showing that the mutant has a single base deletion at position 461 of the wild type sequence.

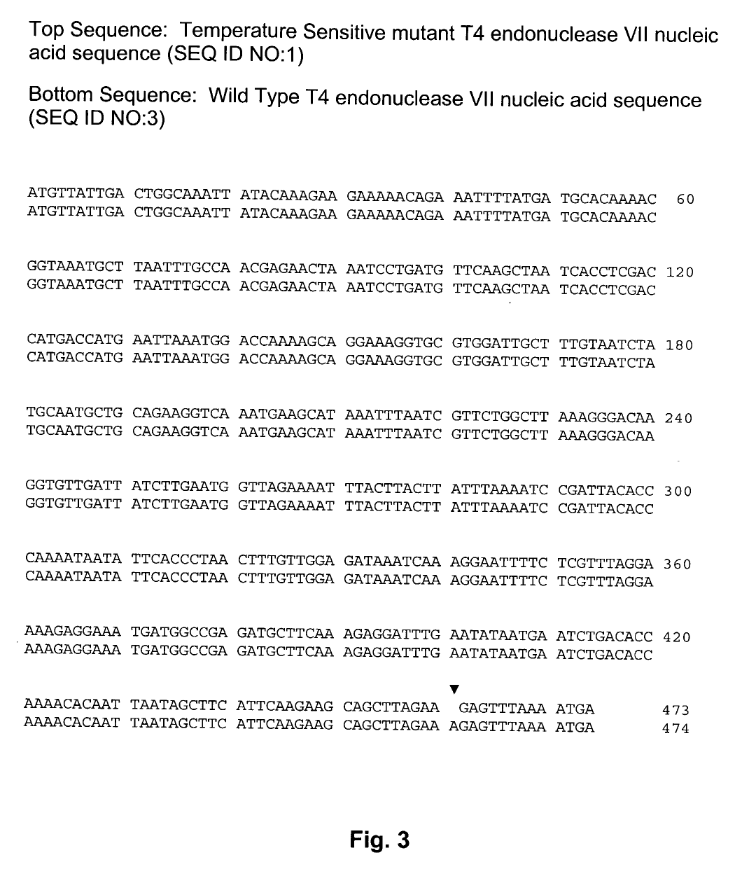

FIG. 4 is a diagram showing the alignment of the amino acid sequences of the temperature-sensitive mutant T4 endonuclease VII nucleic acid sequence (SEQ ID NO: 2) and the wild type T4 endonuclease VII amino acid sequence (SEQ ID NO: 4), showing that the mutant protein is missing the last two amino acids of the wild type protein, and that the last two amino acids of the mutant protein are substituted relative to the corresponding two amino acids in the wild-type protein.

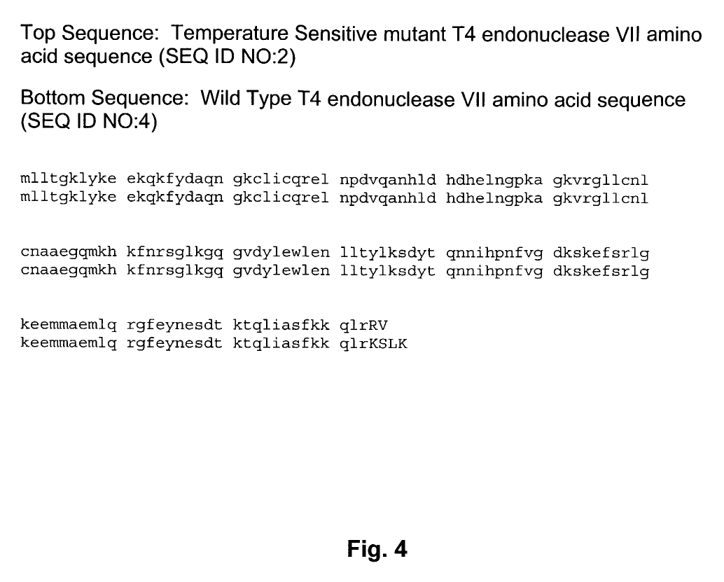

### DETAILED DESCRIPTION

The subject invention provides for a host strain that contains a temperature sensitive variant of the gene encoding the endonuclease VII from phage T4. Using this host strain, the invention features a novel cloning method that selects for PCR products that do not contain PCR generated mutations.

In one embodiment, the invention provides for competent host cells which specifically destroy and mismatched nucleic acids that they contain. These cells contain a plasmid containing an exogenous, conditionally-active resolvase, e.g., a T4 endonuclease VII enzyme which has been mutated to be conditionally active. Preferably, the enzyme has been mutated to be temperature-sensitive, e.g., to be active when the cells are grown at normal temperatures, and inactive when the cells are grown at non-permissive (e.g., elevated) temperatures. Alternatively, a true conditionally-expressed resolvase can be used, that is, a resolvase the expression of which is completely repressed under certain conditions.

The normal T4 endonuclease VII enzyme cannot be expressed and stably maintained in E. coli because it is lethal to those cells into which it is produced. This enzyme is a resolvase, and cleaves double stranded DNA a few bases downstream of any perturbation in the DNA. It therefore destroys nucleic acids in which the two complementary strands are not perfectly matched. E. coli possesses its own DNA repair enzymes, but T4 endonuclease VII, when cloned into E. coli, produces a double strand blunt cleavage at mismatches, and such cleavages cannot be repaired. Such mismatches arise due to replication errors. Therefore, when normal (i.e., unmutated) T4 endonuclease VII is cloned into E. coli and expressed, it destroys the host cell's DNA immediately after it has been synthesized.

In vitro, T4 endonuclease VII cleaves mismatched DNA, but will also digest homoduplex DNA at a reduced level. It is therefore not useful for destroying mismatched DNA in vitro. In vivo, the enzyme's specificity is much higher.

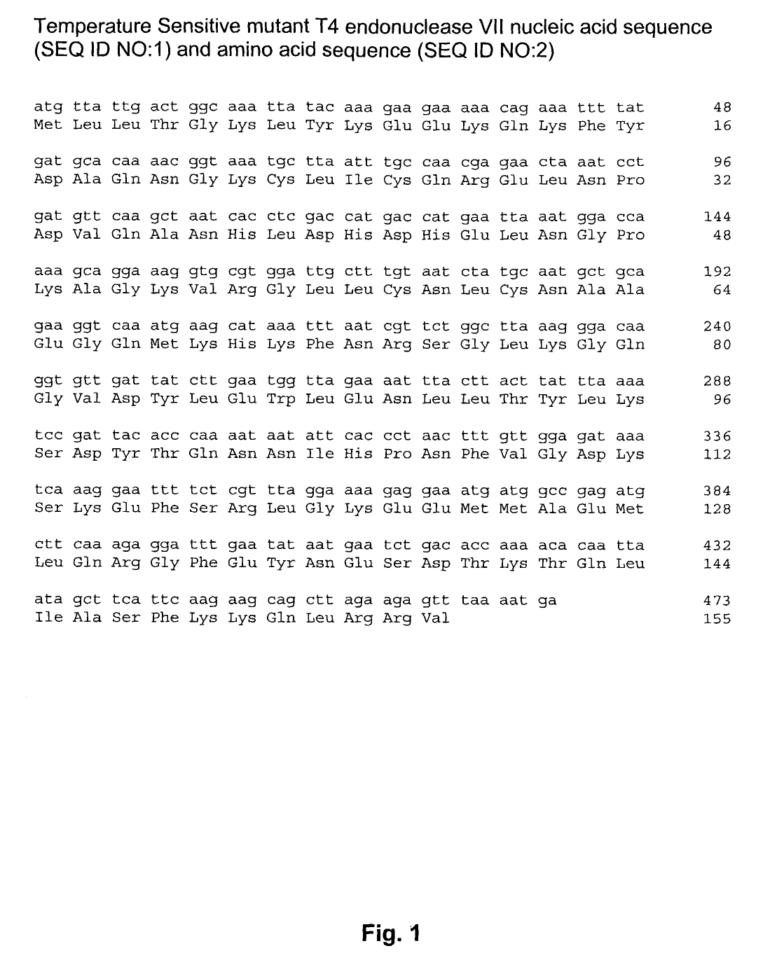

In the host cells of the present invention, a mutated T4 endonuclease VII gene has been introduced into E. coli cells. This mutated enzyme gene encodes an altered endonuclease enzyme that contains a temperature-sensitive mutation, so that the enzyme is active at permissive (e.g., 25° C.) temperatures, but exhibits little or no activity when the host cells are grown at higher temperatures (e.g., 37° C.-42° C.). In the mutated enzyme shown in FIG. 1 (SEQ ID NO: 2), the enzyme is actually expressed when the host cells are grown or maintained at higher temperatures, but the protein folds incorrectly, resulting in little or no activity at those temperatures.

Once the gene encoding the mutated endogenous endonuclease has been transformed into one or more host cells, the cells are grown at the non-permissive temperature, and rendered competent via any methods known in the art. The cells are then briefly maintained at the permissive temperature for 0-3 hours to allow the cell(s) to produce active exogenous resolvase. The cells are then frozen and stored, or are used immediately.

Double-stranded nucleic acids (e.g., amplification products) from which one wishes to select perfectly matched products (e.g., homoduplexes) are then ligated into an appropriate plasmid. The host cells are then thawed (if necessary), and are transformed with the plasmids containing the double-stranded nucleic acid. The active resolvase the acts to degrade any nucleic acid containing a perturbation, e.g., a mismatch.

One can rescue those plasmids containing homoduplexes by use of an F1 helper phage. Such a phage excises plasmids, packages the DNA into a phage particle, and infects the phage-plasmid hybrid molecules (called phagemids) into another F strain of bacteria. Preferably, cells of this other F strain of bacteria are also in the mixture of competent cells, so that the transfer (i.e., the rescue) of the plasmids can be done immediately.

In vitro amplification of nucleic acids (e.g., by polymerase chain reaction) tends to be highly error-prone. If a mutation is induced in a very early cycle, it is possible that the final amplification products will include a high proportion of perfectly matched products, which are yet mutated relative to the original template nucleic acid. The possibility of such an occurrence can be reduced by denaturing and reannealing the final PCR products, to increase the number of mismatches available for action by the resolvase. The number of cloned products will perforce be reduced, but this method increases the chances of removing “false” homoduplexes.

The practice of the present invention will employ, unless otherwise indicated, conventional techniques of molecular biology, cell biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); (Harlow, E. and Lane, D.) Using Antibodies: A Laboratory Manual (1999) Cold Spring Harbor Laboratory Press; and a series, Methods in Enzymology (Academic Press, Inc.); Short Protocols In Molecular Biology, (Ausubel et al., ed., 1995). All patents, patent applications, and publications mentioned herein, both supra and infra, are hereby incorporated by reference in their entirety.

The invention provides a method for the cloning of a PCR product that is essentially free of PCR induced mutations. The invention therefore provides for primers specific for a test DNA template, methods of primer synthesis, methods of DNA amplification and cloning into a phagemid cloning vector. The invention also features host cells that harbor an expression vector containing a temperature sensitive mutant of T4 endonuclease VII. The method therefore provides for methods of mutagenesis and screening procedures required for the identification and isolation of host cells containing a temperature sensitive mutant of T4 endonuclease VII. The invention also provides protocols for the generation of competent host cells, a denaturation/renaturation of the ligated DNA, transformation of the competent host cells with the mixture of DNA homoduplex and heteroduplex molecules, maintenance of the host cells at a temperature permitting the expression of functional T4 endonuclease VII and subsequent rescue by f1 helper phage.

Primers According to the Invention

The invention provides for oligonucleotide primers useful for amplifying DNA or RNA sequences.

Primer Design

Primers may be selected manually by analyzing the template sequence. Computer programs, however, are also available in selecting primers to generate an amplified product with a designed length, e.g., primer premier 5 (available at the website of the company Premierbiosoft) and primer3 (available at the website of the Whitehead Institute for Biomedical Research, Cambridge, Mass., U.S.A).

It is known in the art that primers that are about 20-25 bases long and with 50% G-C content will work well at annealing temperature at about 52-58° C. These properties are preferred when designing primers for the subject invention. Longer primers, or primers with higher G-C contents, have annealing optimums at higher temperatures; similarly, shorter primers, or primers with lower G-C contents, have optimal annealing properties at lower temperatures. A convenient, simplified formula for obtaining a rough estimate of the melting temperature of a primer 17-25 bases long is as follows:

Melting temperature (Tm in ° C.)=4×(# of G+# of C)+2×(# of A+# of T)

Shorter fragments are amplified more efficiently than longer fragments although target of more than 10 kb can be successfully amplified. Preferably the primers are chosen so as to amplify an entire coding region.

In accordance with the preferred embodiments, optimal results have been obtained using primers, which are 19-25 in length. However, one skilled in the art will recognize that the length of the primers used may vary. For example, it is envisioned that shorter primers containing at least 15, and preferably at least 17, may be suitable. The exact upper limit of the length of the primers is not critical. However, typically the primers will be less than or equal to approximately 50 bases, preferably less than or equal to 30 bases.

Primer Synthesis

Methods for synthesizing primers are available in the art. The oligonucleotide primers of this invention may be prepared using any conventional DNA synthesis method, such as, phosphotriester methods such as described by Narang et al. (1979, Meth. Enzymol. 68:90) or Itakura (U.S. Pat. No. 4,356,270), or and phosphodiester methods such as described by Brown et al. (1979, Meth. Enzymol. 68:109), or automated embodiments thereof, as described by Mullis et al. (U.S. Pat. No. 4,683,202). Also see particularly Sambrook et al. (1989), Molecular Cloning: A Laboratory Manual (2d ed.; Cold Spring Harbor Laboratory: Plainview, N.Y.), herein incorporated by reference in its entirety.

Useful DNA Polymerases and Reverse Transcriptases

DNA polymerases and their properties are described in detail in, among other places, DNA Replication 2nd edition, Komberg and Baker, W. H. Freeman, New York, N.Y. (1991).

Known conventional DNA polymerases include, for example, Pyrococcus furiosus (Pfu) DNA polymerase (Lundberg et al., 1991, Gene, 108:1, provided by Stratagene, La Jolla, Calif., USA), Pyrococcus woesei (Pwo) DNA polymerase (Hinnisdaels et al., 1996, Biotechniques, 20:186-8, provided by Boehringer Mannheim, Roche Molecular Biochemicals, Indianapolis, Ind., USA), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Polynucleotides Res, 19: 4193, provided by New England Biolabs, Beverly, Mass., USA), 9°Nm DNA polymerase (discontinued product from New England Biolabs, Beverly, Mass., USA), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), Pyrococcus kodakaraensis KOD DNA polymerase (Takagi et al., 1997, Appl. Environ. Microbiol. 63:4504), JDF-3 DNA polymerase (from Thermococcus sp. JDF-3, Published International patent application WO 0132887), Pyrococcus GB-D (PGB-D) DNA polymerase (also referred as Deep-Vent DNA polymerase, Juncosa-Ginesta et al., 1994, Biotechniques, 16:820, provided by New England Biolabs, Beverly, Mass., USA), UlTma DNA polymerase (from thermophile Thermotoga maritima; Diaz and Sabino, 1998 Braz. J. Med. Res. 31:1239; provided by PE Applied Biosystems, Foster City, Calif., USA), Tgo DNA polymerase (from Thermococcus gorgonarius, provided by Roche Molecular Biochemicals, Indianapolis, Ind., USA), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1/DP2 DNA polymerase II (Cann et al., 1998, Proc Natl Acad Sci USA 95:14250-5). The polymerization activity of any of the above enzymes can be defined by means well known in the art. One unit of DNA polymerization activity of conventional DNA polymerase, according to the subject invention, is defined as the amount of enzyme which catalyzes the incorporation of 10 nmoles of total deoxynucleotides (dNTPs) into polymeric form in 30 minutes at optimal temperature (e.g., 72° C. for Pfu DNA polymerase). Assays for DNA polymerase activity and 3′-5′ exonuclease activity can be found in DNA Replication 2nd Ed., Komberg and Baker, supra; Enzymes, Dixon and Webb, Academic Press, San Diego, Calif. (1979), as well as other publications available to the person of ordinary skill in the art.

When using the subject compositions in reaction mixtures that are exposed to elevated temperatures, e.g., during the PCR technique, use of thermostable DNA polymerases is preferred.

Reverse transcriptases useful according to the invention include, but are not limited to, reverse transcriptases from HIV, HTLV-1, HTLV-II, FeLV, FIV, SIV, AMV, MMTV, MoMuLV and other retroviruses (for reviews, see for example, Levin, 1997, Cell 88:5-8; Verma, 1977, Biochim. Biophys. Acta 473:1-38; Wu et al., 1975, CRC Crit. Rev. Biochem. 3:289-347).

Phagemid Cloning Vectors Useful According to the Invention.

Methods well known to those skilled in the art can be used to construct phagemid cloning vectors containing a polynucleotide of the invention. These methods include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989).

In a preferred embodiment, the invention provides for phagemid cloning vectors that typically contain an origin of DNA replication, e.g., a colE1 origin or any of a number of plasmid origins of replication, and also a F1 origin of replication that enables phage controlled DNA replication. A number of phagemid vectors are commercially available such as the pBluescript II phagemids (Stratagene Catalog #212205, #212206, #212207 and #212208) which has an extensive polylinker with 21 unique restriction enzyme recognition sites. Flanking the polylinker are T7 and T3 RNA polymerase promoters that can be used to synthesize RNA in vitro. pBluescript II phagemids contain a 454-bp filamentous f1 phage intergenic region (M13 related), which includes the 307-bp origin of replication. The (+) and (−) orientations of the f1 intergenic region allow the rescue of sense or antisense ssDNA by a helper phage which promotes the packaging of the replicated single stranded phagemid DNA into infectious phage particles that are secreted into the media. Phagemids therefore permit the rescue of cloned sequences without the need for traditional subcloning methods.

Expression Vectors According to the Invention

The invention provides for vectors for the expression of variants of endonuclease VII of phage T4. Appropriate cloning and expression vectors for use with prokaryotic and eukaryotic hosts are described by Sambrook et al., in Molecular Cloning: A Laboratory Manual, Second Edition, Cold Spring Harbor, N.Y. (1989), the disclosure of which is incorporated herein by reference in its entirety.

The DNA sequence in the expression vector is operatively linked to an appropriate expression control sequence(s) (promoter) to direct MRNA synthesis. Examples of such promoters include but are not limited to: LTR or SV40 promoter in mammalian cells, the E. coli. lac or trp, the phage P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain a gene to provide a phenotypic trait for selection of transformed host cells such as dihydrofolate reductase or neomycin resistance for eukaryotic cell culture, or such as tetracycline or ampicillin resistance in E. coli. The vector containing the appropriate DNA sequence as hereinabove described, as well as an appropriate promoter or control sequence, may be employed to transform an appropriate host to permit the host to express the protein.

In a preferred embodiment, the variant T4 endonuclease VII genes are cloned into a pACYC 184 expression vector that contains a multiple cloning site (MCS), the lac operator, and P15A that is compatible plasmids containing a colE1 origin of replication (Nakano et al.(1995) Gene 162: 157-158).

Host Cells Useful According to the Invention

The present invention further provides host cells containing the vectors of the present invention, wherein the nucleic acid has been introduced into the host cell using known transformation, transfection or infection methods. The host cell can be a eukaryotic host cell, such as a mammalian cell, a plant cell, a lower eukaryotic host cell, such as a yeast cell, or the host cell can be a prokaryotic cell, such as a bacterial cell.

Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

In yeast, a number of vectors containing constitutive or inducible promoters may be used. For a review see, Current Protocols in Molecular Biology, Vol. 2, Ed. Ausubel et al., Greene Publish. Assoc. & Wiley Interscience, Ch. 13 (1988); Grant et al. (1987) “Expression and Secretion Vectors for Yeast”, Methods Enzymol. 153:516-544; Glover, DNA Cloning, Vol. II, IRL Press, Wash., D.C., Ch. 3 (1986); Bitter, Heterologous Gene Expression in Yeast, Methods Enzymol. 152:673-684 (1987); and The Molecular Biology of the Yeast Saccharomyces, Eds. Strathem et al., Cold Spring Harbor Press, Vols. I and II (1982).

In a preferred embodiment, the host of the invention is a prokaryotic cell such as E. coli, other enterobacteriaceae such as Salmonella typhimurium, bacilli, various pseudomonads, or other prokaryotes which can be transformed, transfected, and/or infected. The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell, which drives expression of the polynucleotides in the cell.

In another preferred embodiment, the host strain XL1-Blue MRF [Stratagene (Catalog #200301); Genotype: k(mcrA)183 k(mcrCB-hsdSMR-mrr)173 endA1 supE44 t hi-1 recA1 gyrA96 relA1 lac [F proAB1 lacI<ZkM15 Tn10 (Tet′)] is used for the propagation of pBluescript II phagemids and for transformation of recombinant phagemids.

In another preferred embodiment, the host cell conditionally expresses one or more resolvases of prokaryotic or eukaryotic origin.

In another preferred embodiment, the host cell of the invention is XL1-Blue MRF that conditionally expressed a variant of the endonuclease VII of T4 phage.

In another preferred embodiment, the host cell of the invention is XL1-Blue MRF that conditionally expressed a temperature-sensitive mutant of the endonuclease VII of T4 phage.

Generation of Conditionally Expressed Endonuclease VII of Phage T4

The T4 endonuclease VII gene product, if expressed in E. coli, is lethal to the host cells since during the normal bacterial life cycle, replication errors become sites for endonuclease cleavage and ultimately leads to the destruction of the chromosomal integrity and cell death. To overcome the inherent toxicity, the T4 endonuclease VII gene is randomly mutagenized, cloned into an expression vector and then transformed into an appropriate host cell such as XL1-Blue MRF. Transformants are then screened for the presence of conditional lethal mutations within the T4 endonuclease VII open reading frame that permit cell growth at permissive conditions but not a non-permissive conditions.

Mutagenesis

Methods of random mutagenesis that generate one or more randomly-situated mutations are known in the art. One example of such a method is to clone the sequence of interest into a strain of E. coli that has a high spontaneous mutation rate (i.e., a “mutator strain”, e.g., E. coli strain XL1-RED). It was this method that was used to generate the mutant T4 endonuclease VII of the present invention.

An example of a method for random mutagenesis is the so-called “error-prone PCR method”. As the name implies, the method amplifies a given sequence under conditions in which the DNA polymerase does not support high fidelity incorporation. The conditions encouraging error-prone incorporation for different DNA polymerases vary, however one skilled in the art may determine such conditions for a given enzyme. A key variable for many DNA polymerases in the fidelity of amplification is, for example, the type and concentration of divalent metal ion in the buffer. The use of manganese ion and/or variation of the magnesium or manganese ion concentration may therefore be applied to influence the error rate of the polymerase. The resulting PCR product is cloned into multiple cloning site downstream of the IPTG inducible lactose promoter of the expression vector, pACYC 184 using standard recombinant DNA techniques that are known to one of skill in the art (see FIG. 1). The recombinant expression vector is then transformed into competent XL1-Blue MR and plated out on Luria broth plates containing chloramphenicol (50 μg/ml) according to standard procedure.

Screening for Temperature Sensitive Mutants of T4 Endonuclease VII

Chloramphenicol resistant colonies are then screened in the presence of the IPTG inducer (0.1-1 mM) for mutants of T4 endonuclease VII that are temperature sensitive. These mutants grow normally at temperatures of 37° C. and above, where the enzyme is inactive, but are incapable of growth at room temperature whether the T4 endonuclease expression was induced or not. The nucleotide sequence of the temperature sensitive mutant of T4 endonuclease VII is provided in SEQ ID NO: 1. The wild type version is shown in SEQ ID NO: 2. Host cells carrying the temperature sensitive T4 endonuclease VII gene can then be propagated at the permissive temperature of 37° C.

Generation of Competent Cells

Prior to making these cells competent for transformation (chemical or electrocompetent) expression of the temperature sensitive T4 endonuclease VII enzyme is turned on by addition of IPTG inducer (1 mM) and the inactive enzyme reactivated by its presumed correct folding at temperatures below 37° C. (see Example 2, below).

Methods of generating competent cells are well known to those skilled in the art. Typical methods of generating competent cells comprise growing cells to log phase or early stationary phase and exposing the cells to CaCl₂ or other cationic compound (see, e.g., Sambrook et al., In Molecular Cloning: a Laboratory Manual, 2nd Edition, eds. Sambrook et al., Cold Spring Harbor Laboratory Press, (1989)). Cells can be contacted immediately with exogenous DNA or frozen in glycerol or DMSO for subsequent use. Upon thawing to 4° C. and contacting with plasmid DNA, frozen competent cells typically have transformation efficiencies of 1×10⁵-1×10⁹ transformants/μg of plasmid DNA.

Generation of Electrocompetent Cells

Electroporation has also been used to transform cells (see, e.g., Dower et al., Nucleic Acids Research 16:6127-6145 (1988); Taketo, Biochimica et Biophysica Acta 949:318-324 (1988); Chassy and Flickinger, FEMS Microbiology Letters 44:173-177 (1987); and Harlander, Streptococcal Genetics, eds. Ferretti and Curtiss, American Society of Microbiology, Washington, D.C., pp. 229-233 (1987)). Electroporation methods rely on creating temporary holes in cell membranes by exposing cells to a high voltage electric impulse to facilitate the uptake of exogenous nucleic acids (see, e.g., Andreason and Evans, Biotechniques 6:650-660 (1988)). Cells exposed to an electroporation buffer (e.g., 10-15% glycerol) are generally stored by freezing to provide a supply of electrocompetent cells (see, e.g., U.S. Pat. No. 6,004,804).

DNA Denaturation and Renaturation According to the Invention

The formation of a duplex is accomplished by denaturing and then annealing two homologous and complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially, or completely, complementary.

Preferably, single-stranded DNA is prepared by denaturing double-stranded test DNA in distilled water with heat (i.e. between 90° C. and 100° C.). Those skilled in the art will appreciate that DNA denaturation can also be accomplished by heat denaturation, followed by renaturation at progressively lower temperatures. Heteroduplex formation between the different strands of a heat denatured test DNA is performed in 50 μl (total volume) containing 1 times annealing buffer as previously described (Cotton, Methods in Molecular Biology 9:39 (1991)) except that the annealing temperature is set at 65° C. for 1 hour followed by 20 min at room temperature.

Phage Rescue According to the Invention

M13KO7 (Stratagene (La Jolla, Calif., USA) Catalog No.: #N0315S) is an M13 derived helper phage that carries the mutation Met40IIe in gII, the origin of replication from P15A and the kanamycin resistance gene from Tn903 both inserted within the M13 origin of replication. M13KO7 is able to replicate in the absence of phagemid DNA. In the presence of a phagemid bearing a wild-type M13 or f1 origin such as pBluescript II, single-stranded phagemid is packaged preferentially and secreted into the culture medium. Since these filamentous helper phages (M13, f1) will not infect E. coli without an F episome coding for pili, it is essential to use XL1-Elue MRF or a similar strain containing the F episome. Typically, 30-50 pBluescript II molecules are packaged/helper phage DNA molecule. pBluescript II phagemids are offered with the IG region in either of two orientations: pBluescript II (+) is replicated such that the sense strand of the β-galactosidase gene is secreted within the phage particles; pBluescript II (−) is replicated such that the antisense strand of the β-galactosidase gene is secreted in the phage particles. Yields of single-stranded (ss)DNA depend on the specific insert sequence. For most inserts, over 1 pg of ssDNA can be obtained from a 1.5-ml miniculture.

Generation of Competent Cells Harboring a Temperature Sensitive T4 Endonuclease VII

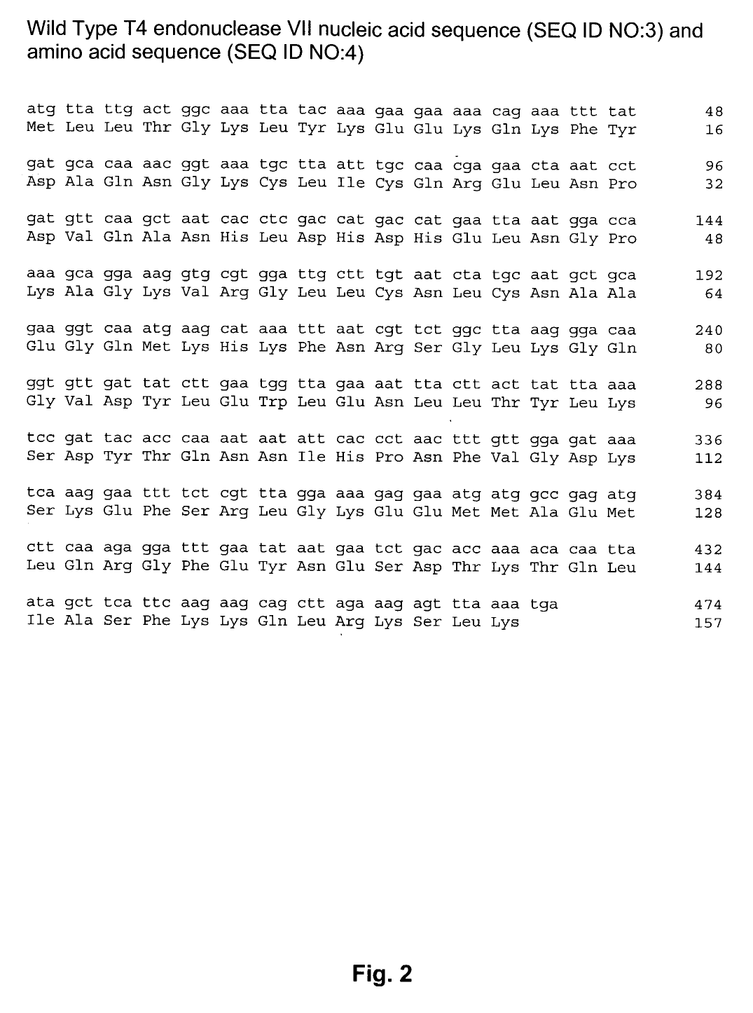

In vitro experiments demonstrate that T4 endonuclease VII not only digests heteroduplex DNA at the sites of mismatch but also homoduplex DNA at random (but reproducible) sites. These preliminary experiments thus established that, unless other discriminatory factors are discovered, T4 endonuclease VII can only be used in an in vivo setting, where the accuracy of the enzyme ensures that homoduplex DNA is never cleaved. However, the T4 endonuclease gene product, if expressed in E. coli, is lethal to the host cells since during the normal bacterial life cycle, replication errors (which are normally repaired post-replicationally) become sites for endonuclease cleavage and leads to the destruction of the chromosomal integrity and eventual cell death. To overcome this problem, T4 endonuclease VII was mutagenized (see above), cloned into an pACYC expression vector downstream of the lactose promoter and transformed into a XL1-Blue MRF containing a pCALnEK plasmid that expresses the lactose high affinity repressor (lacI^{q}) and a second expression plasmid (R6K) that enhances the repression in this setting (see FIG. 2). Transformants were then screened for the presence of temperature sensitive mutations and a conditional lethal mutant was isolated that grows normally at temperatures above 37° C. (enzyme inactive) but were incapable of growth at room temperature whether the T4 endonuclease expression was induced or not.

Chemical methods of making competent cells as well as electrocompetent cells are well known in the art cells (see U.S. Pat. Nos. 5,512,468; 6,338,965 and 6,040,184 and references cited therein). Prior to DNA transformation, expression of the temperature sensitive T4 endonuclease VII can be both turned on and the inactive enzyme reactivated by its presumed correct folding at temperatures below 37° C. During this relatively brief period, the E. coli host cells have not yet died (and thus can be transformed with plasmid DNA) but will contain active endonuclease to cleave incoming DNA containing a mismatch.

PCR Amplification and Ligation

Methods for PCR cloning of a target DNA sequence are well known to those skilled in the art. A number of kits are now commercially available that provide all reagents and reaction conditions for the generation of PCR DNA fragments and subsequent cloning into a phagemid cloning vector. In one embodiment of the present invention, PCR fragments are generated using a TOPO cloning kit (InVitrogen, San Diego, Calif., USA), PCR-Script® cloning kit (Stratagene (La Jolla, Calif., USA); catalog #211188), or other method or kit. PCR products are incubated with one of the predigested PCR-Script phagemid cloning vectors, SrfI and T4 DNA ligase. Using the restriction enzyme in the ligation reaction maintains a high-steady-state concentration of digested vector DNA and allows the use of nonphosphorylated, unmodified PCR primers. The ligation efficiency of blunt-ended DNA fragments is increased by the simultaneous, opposite reactions of the SrfI restriction enzyme and T4 DNA ligase on nonrecombinant vector DNA. The PCR-Script cloning kits allow rapid and efficient cloning of all PCR products, regardless of the PCR enzyme used to generate the inserts. PCR-Script kits also include the StrataPrep® PCR purification kit for easy and efficient removal of primers and nonspecific amplification products smaller than 100 bp.

The PCR product is diluted with distilled water and heat denatured and renatured before it is ligated to the vector. Induced competent host cells containing activated temperature sensitive T4 endonuclease VII encoded by SEQ ID NO: 1 are then added to the heat denatured, renatured PCR ligation mix. Once the DNA to be transformed has entered the cells that express T4 endonuclease VII, those DNA molecules that contain mutations are cleaved by the active T4 endonuclease VII whereas those DNA molecules that are mutation free are not digested by endonuclease VII. Since these host cells will eventually die due to the lethality of T4 endonuclease VII, the non mutant DNAs are rescued In a first preferred embodiment, the host cells are infected with a f1 helper phage that encodes the enzymes necessary to replicate plasmids containing a f1 origin or replication such as pUC and pBluescript and the like. This replication is followed by packaging of a single stranded molecule that can then infect a secondary host strain that harbors the F′ episome Incoming DNA molecules that harbor mutations and are cleaved by T4 endonuclease VII are unable to be packaged. In another embodiment, non-mutant plasmids are rescued by conjugal mating. In this scenario, the incoming plasmid DNA molecule should contain an origin of conjugal transfer (oriT; Ditta, G. et. al., Proc. Natl. Acad. Sci. USA 77:7347-51, 1980; Hengen, P. N. and V. N. Iyer, Biotechniques 13:56-62 (1992)) and a second expression plasmid encoding the conjugal transfer proteins.

Kits

The invention is intended to provide novel compositions and methods for the cloning of PCR amplification products devoid of PCR-induced mutations, as described herein. The invention herein also contemplates a kit format that comprises a package unit having one or more containers of the subject composition and in some embodiments including containers of various reagents used for polynucleotide synthesis, including synthesis in PCR. The kit may also contain one or more of the following items: polymerization enzymes, dNTPs, primers, buffers, antibiotics, helper phage, instructions, and controls. The Kits may include containers of reagents mixed together in suitable proportions for performing the methods in accordance with the invention. Reagent containers preferably contain reagents in unit quantities that obviate measuring steps when performing the subject methods. In one embodiment, the kit contains XL1-Blue MRF cells containing a pCALnEK plasmid that expresses the lactose high affinity repressor (lacI^{q}), a second expression plasmid (R6K) that enhances the repression and pACYC 184 expression plasmid containing SEQ ID NO: 1, which encodes the temperature sensitive T4 endonuclease VII.

### EXAMPLES

### Example 1

Preparation of Plasmid Ligation Mixture.

The amplified PCR product of interest is separated from the PCR amplification enzyme by any conventional method (i.e., resin or spin cup to remove enzyme—the manual for a Stratagene product to perform this operation will be included in the additional materials).

The DNA is then heated to 95° C. for 5 minutes, then allowed to slowly cool at room temperature (or in a thermal heating block whose temperature can be ramped down) until the mixture reaches room temperature. This material is then ligated or annealed to the plasmid of interest. This DNA can also be transformed directly into a host cell, depending on the application and the ultimate goal of the experiment. For instance, the transforming DNA can be recombined with a plasmid that is already present in the host, the DNA may recombine with the chromosomal DNA, the DNA may replicate as a linear molecule, etc.

### Example 2

Preparation of Host Cells.

The E. coli host cells harboring the T4 Endonuclease VII (and the lacI^{q} encoding repressor plasmid, the lacI^{q} enhancer plasmid and the F′ episome) are grown at 37° C. or above until the cells reach an optical density of 0.2 (at 550 nm). The inducer molecule (IPTG) is added and the cells are maintained at 42° C. for 30 minutes. The cells are rapidly brought to room temperature and are maintained there for a period of 0-3 hours. These cells are then made competent by standard methods (chemically competent or electrocompetent) and frozen at −80° C. prior to use.

These “competent” cells are then thawed, an appropriate volume withdrawn and the plasmid ligation mixture added. Transformation protocol is standard. After heat pulse or electric shock, f1 helper phage (containing the enzymes necessary to replicate and package plasmid DNA molecules containing the f1 phage origin of replication) is added at the same time as the terminal acceptor strain (any E. coli containing the F′ episome required for f1 infection). The f1 helper phage will package any plasmid DNA molecule containing the f1 origin of replication—those that have been subject to T4 endonuclease VII will not be contiguous and cannot be packaged. The packaged phagemid is then readily transferred to the terminal host (see diagram).

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A host cell for selectively cloning homoduplex nucleic acid molecules, wherein the host cell is:
(a) competent;
(b) contains a heterologous gene encoding a resolvase that is conditionally expressed and/or conditionally active; and
(c) can be maintained under conditions under which the resolvase is not expressed or repressed and propagated in inactive form.

2. The host cell of claim 1, wherein said host cell is eukaryotic.

3. The host cell of claim 1, wherein said host cell is prokaryotic.

4. The host cell of claim 1, wherein said conditionally expressed resolvase is selected from the group consisting of: a bacteriophage resolvase, a prokaryotic resolvase, and a eukaryotic resolvase.

5. The host cell of claim 1, wherein said conditionally expressed resolvase is bacteriophage T4 Endonuclease VII.

6. The host cell of claim 1, wherein said conditionally expressed resolvase is a temperature sensitive mutant of bacteriophage T4 Endonuclease VII.

7. The host cell of claim 1, wherein said conditionally expressed resolvase is a temperature sensitive mutant of bacteriophage Endonuclease VII encoded by SEQ ID NO: 1.

8. A method of selectively cloning homoduplex nucleic acid molecules, the method comprising:
(a) providing one or more host cells containing one or more heterologous genes, where each gene encodes a conditionally expressed resolvase;
(b) transforming said host cells with cloned nucleic acid molecules, thereby producing transformed host cells; and
(c) maintaining the transformed host cells under conditions suitable for expression of the one or more resolvases, wherein, within a host cell containing a heteroduplex cloned nucleic acid molecule, the heteroduplex cloned nucleic acid molecule is destroyed by the resolvase, and within a host cell containing a homoduplex cloned nucleic acid molecule, the homoduplex cloned nucleic acid molecule is not destroyed by the resolvase;
thereby selectively cloning homoduplex nucleic acid molecules.

9. The method of claim 8, further comprising the step of, before (b), denaturing and renaturing the nucleic acid molecules.

10. The method of claim 8, further comprising the step of, after (e), infecting said cells with helper phage, thereby rescuing the homoduplex cloned nucleic acid molecules.

11. The method of claim 8, further comprising the step of maintaining the one or more host cells under conditions that prohibit the expression of the one or more resolvases.

12. The method of claim 8, wherein said host cell is eukaryotic.

13. The method of claim 8, wherein said host cell is prokaryotic.

14. The method of claim 8, wherein said cloning vector is a phagemid cloning vector.

15. The method of claim 8, wherein said one or more conditionally expressed resolvase is selected from the group consisting of: a bacteriophage resolvase, a prokaryotic resolvase, and a eukaryotic resolvase.

16. The method of claim 8, wherein said one or more conditionally expressed resolvases is bacteriophage T4 Endonuclease VII.

17. The method of claim 8, wherein said one or more conditionally expressed resolvases is a temperature sensitive mutant of bacteriophage T4 Endonuclease VII.

18. The method of claim 8, wherein said one or more conditionally expressed resolvases is the temperature sensitive mutant of bacteriophage Endonuclease VII encoded by SEQ ID NO: 1.

19. A method of selectively cloning amplified nucleic acid molecules possessing a reduced number of mutations, the method comprising:
(a) providing one or more host cells containing one or more heterologous genes, wherein each gene encodes a conditionally expressed resolvase;
(b) transforming said host cells with cloned amplified nucleic acid molecules, thereby producing transformed host cells; and
(c) maintaining the transformed host cells under conditions suitable for expression of the one or more resolvases, wherein, within a host cell containing a heteroduplex cloned nucleic acid molecule, the heteroduplex cloned nucleic acid molecule is destroyed by the resolvase, and within a host cell containing a homoduplex cloned nucleic acid molecule, the homoduplex cloned nucleic acid molecule is not destroyed by the resolvase;
thereby selectively cloning amplified nucleic acid molecules possessing a reduced number of mutations.

20. The method of claim 19, further comprising the step of, before (b), denaturing and renaturing the nucleic acid molecules.

21. The method of claim 19, further comprising the step of, after (e), infecting said cells with helper phage, thereby rescuing the homoduplex ligated nucleic acid molecules.

22. The method of claim 19, further comprising the step of maintaining the one or more host cells under conditions that prohibit the expression of the one or more resolvases.

23. The method of claim 19, wherein said host cell is eukaryotic.

24. The method of claim 19, wherein said host cell is prokaryotic.

25. The method of claim 19, wherein said cloning vector is a phagemid cloning vector.

26. The method of claim 19, wherein said one or more conditionally expressed resolvase is selected from the group consisting of: a bacteriophage resolvase, a prokaryotic resolvase, and a eukaryotic resolvase.

27. The method of claim 19, wherein said one or more conditionally expressed resolvases is bacteriophage T4 Endonuclease VII.

28. The method of claim 19, wherein said one or more conditionally expressed resolvases is a temperature sensitive mutant of bacteriophage T4 Endonuclease VII.

29. The method of claim 19, wherein said one or more conditionally expressed resolvases is the temperature sensitive mutant of bacteriophage Endonuclease VII encoded by SEQ ID NO: 1.

30. A kit comprising a competent host cell containing a heterologous gene encoding a conditionally expressed resolvase, and packaging materials therefore.

31. A kit comprising a competent host cell containing a heterologous gene encoding a conditionally expressed resolvase, wherein the host cell can be maintained under conditions under which the resolvase is expressed and packaging materials therefore.

32. A kit comprising a host cell containing an expression vector encoding a temperature sensitive mutant of bacteriophage T4 Endonuclease VII of SEQ ID NO: 2, PCR reagents, helper phage and packaging materials therefore.

33. A kit comprising an isolated DNA encoding the temperature sensitive mutant of bacteriophage T4 Endonuclease VII of SEQ ID NO: 2 and packaging materials therefore.

34. A kit comprising an expression vector encoding a temperature sensitive mutant of bacteriophage T4 Endonuclease VII of SEQ ID NO: 2 and packaging materials therefore.

35. A kit comprising a host cell containing an expression vector encoding a temperature sensitive mutant of bacteriophage T4 Endonuclease VII of SEQ ID NO: 2 and packaging materials therefore.

36. An isolated nucleic acid encoding a temperature sensitive mutant of bacteriophage T4 Endonuclease VII, wherein said isolated nucleic acid has the nucleic acid sequence of SEQ ID NO: 1.

37. The isolated nucleic acid of claim 37, wherein said isolated nucleic acid is cloned into an expression vector.

38. An isolated protein of a temperature sensitive mutant of bacteriophage T4 Endonuclease VII, wherein said isolated protein has the amino acid sequence of SEQ ID NO: 2.
